(19) United States
(12) Patent Application Publication
(10) Pub. No.: US 2010/0016550 A1
(43) Pub. Date: Jan. 21, 2010

(54) Methods for Site-Specific Pegylation

(76) Inventors: Zheng Xin Dong, Holliston, MA (US); John S. Eynon, Bellingham, MA (US)

Correspondence Address: Alan F Feeney;Patent Department, 27 Maple Street, Milford, MA, 01757-3650, US

(21) Appl. No.: 12/227,760

(22) PCT Filed: May 25, 2007
(86) PCT No.: PCT/US2007/012621

§ 371 Date: Nov. 26, 2008

Related U.S. Application Data
(60) Provisional application No. 60/809,098, filed on May 26, 2006.
(60) Provisional application No. 60/873,494, filed on Dec. 7, 2006.

(51) Int. Cl. C07K 5/08; C07D 277/04; C07D 279/06; C07D 207/40; C07K 5/06

(52) U.S. Cl. 530/331; 548/200; 544/54; 548/546

(57) ABSTRACT

The present invention relates to methods for the chemo-selective pegylation of the cysteine residue having unoxidized sulfhydryl side-chain and free α-amino group in proteins, peptides and other molecules. Similar methods are provided for the chemo-selective pegylation of the homocysteine, selenocysteine, penicillamine, and N-methyl-cysteine residues.

### BACKGROUND OF THE INVENTION

The present invention relates to methods for the chemo-selective pegylation of the cysteine residue having an unoxidized sulfhydryl side-chain and a free α-amino group in proteins, peptides and other molecules.

Unlike small molecule drugs which are usually administered by oral route, protein- and peptide-based therapeutic agents are typically administered by injection due to their extremely low oral bioavailability. After injection most proteins and peptides are rapidly cleaved by enzymes and cleared from the body, resulting in short in vivo circulating half-life. The short circulating half-life is responsible for lower efficacy, more frequent administration, reduced patient compliance, and higher cost of protein and peptide therapeutics. Thus, there is a strong need to develop methods to prolong the duration of action of protein and peptide drugs.

Covalent attachment of proteins or peptides to polyethylene glycol (PEG) has proven to be a useful method to increase the circulating half-lives of proteins and peptides in the body (Abuchowski, A. et al., Cancer Biochem. Biophys., 1984, 7:175-186; Hershfield, M. S. et al., N. Engl. J. Medicine 316:589-596; and Meyers, F. J. et al., Clin. Pharmacol. Ther., 1991, 49:307-313). Covalent attachment of PEG to proteins and peptides not only protects the molecules against enzymatic degradation, but also reduces their clearance rate from the body. The size of PEG attached to a protein has significant impact on the circulating half-life of the protein. Usually the larger the PEG is, the longer the in vivo half-life of the attached protein is. Several sizes of PEGs are commercially available (Nektar Advanced PEGylation Catalog 2005-2006; and NOF DDS Catalogue Ver 7.1), which are suitable for producing proteins and peptides with targeted circulating half-lives. PEG moiety also increases water solubility and decreases immunogenicity of proteins, peptides and other molecules (Katre, N. V. et al., Proc. Natl. Aced. Sci. USA, 1998, 84:1487-1491; and Katre N. V. et al., J. Immunology, 1990, 144:209-213).

Several methods of pegylating proteins have been reported in the literature. For example, N-hydroxy succinimide (NHS)-PEG was used to pegylate the free amine groups of lysine residues and N-terminus of proteins. Because proteins usually contain multiple lysine residues and terminal amine group, multiple sites of a protein are pegylated by using this method. Such non-selective pegylation results in decreasing the potency of the pegylated proteins because multiple PEG moieties usually disturb the interaction between the proteins and their biological target molecules (Teh, L.-C. and Chapman, G. E., Biochem. Biophys. Res. Comm., 1988, 150:391-398; and Clark, R. et al., J. Biol. Chem. 1996, 271:21969-21977). Multiple-site, non-selective pegylation also generates heterogeneous mixtures of final products. Many of these heterogeneous pegylated proteins are not suitable for medical use because of low specific activities. It is difficult to purify and characterize heterogeneous pegylated proteins. The variation of contents between different product batches of heterogeneous pegylated proteins is usually high and quality control on these mixtures is difficult.

Although PEGs bearing aldehyde groups have been used to pegylate the amino-termini of proteins in the presence of a reducing reagent, such a method does not generate exclusive N-terminal pegylated proteins and the lysine residues of the proteins are also pegylated. Thus, the resulting proteins are also heterogeneous mixtures (Kinstler O. B. et al., U.S. application Ser. No. 09/817,725). This method also suffers the drawback of using harsh reduction reaction conditions. The reducing reagents such as cyanoborohydride could harm the proteins and give lower reaction yields.

PEGs with maleimide functional groups were used for selectively pegylating the free thiol groups of cysteine residues in proteins. Such method often requires point mutation with new cysteine. Because most proteins contain one or more cysteine residues, to selectively keep the thiol group of the new, “unnatural” cysteine residue from forming a disulfide bridge with other cysteine residues and then to selectively pegylate that particular new cysteine requires complicated reaction conditions (U.S. Pat. No. 6,753,165, issued Jun. 22, 2004; and U.S. Pat. No. 6,608,183, issued Aug. 19, 2003). Even under the controlled reaction conditions, other cysteine residues can be pegylated and heterogeneous materials are obtained.

Site-specific pegylation of acetyl-phenylalanine residue of growth hormone analogs were reported. Such method requires point mutation with unnatural amino acid acetyl-phenylalanine (U.S. application Ser. No. 11/046,432, filed Jan. 28, 2005). One of the drawbacks of this method is that pegylation of proteins bearing unnatural amino acids, such as acetyl-phenylalanine, can only been done in bacteria but not in mammalian cells.

The free thiol and amine groups generated from the reaction of an amine thiolactone with free amine group of interleukin-2 have been used to pegylate the protein. However, in this method, the amine thiolactone used reacts with any amine functional groups of lysine residues and N-terminus in proteins and the method is not site-selective (U.S. Pat. No. 6,310,180, issued Oct. 30, 2001).

Therefore, despite the previous efforts from different groups, there is still a strong need to develop easy and practical methods for site-specific pegylation of proteins, peptides and other molecules.

### SUMMARY OF THE INVENTION

The present invention generally relates to new methods for site-specific pegylation of proteins, peptides and other molecules. It was discovered that PEG containing an aldehyde functional group (PEG-aldehyde) reacts spontaneously with cysteine bearing an unoxidized sulfhydryl side-chain and a free α-amino group in aqueous solution in a wide range of pHs to generate thiazolidine allowing for PEG-aldehyde to react with a peptide fragment containing variety of functional groups which was not certain due to the hydrophilic nature and large size (e.g., 30 kDa) of PEG. We also discovered that only the cysteine residue having an unoxidized sulfhydryl side-chain and a free α-amino group reacts with PEG-aldehyde. The other functional groups in other residues (e.g., thiol group of cysteine without a free α-amino group, guanidinyl group of Arg, amino group of Lys, side-chain carboxylic acid group of Asp, side-chain carboxylic acid group of Glu, hydroxyl group of Tyr, and hydroxyl group of Ser) do not react with PEG-aldehyde.

By using the present methods, only cysteine residues having an unoxidized sulfhydryl side-chain and a free α-amino group, but not any other amino acids in proteins, peptides and other molecules, are pegylated. Thus, the present methods are highly site-selective. The site-specific nature of the present pegylation methods results in more homogeneous products which are easy to characterize, purify and manufacture and have less content variation between different batches. The PEG attached at a specific site (i.e., N-terminal cysteine) of proteins and peptides should have less chance to interact with the biological targets and should therefore yield more potent therapeutic agents.

In the present invention, the aldehyde functional group of PEG spontaneously reacts with the amine and thiol functional groups of cysteine residue at the N-terminus of protein or peptide in aqueous solution in a range of pH (e.g., pH2-8) and at different temperatures (e.g., room temperature). The newly generated functional group between PEG and protein or peptide is a 1,3-thiazolidine. The carboxy groups of glutamic and aspartic acid residues and the C-terminus carboxy group, the amine groups of lysine residues, guanidinyl groups of arginine residues, thiol groups of middle cysteine residues, and hydroxy groups of serine, threonine and tyrosine residues do not react with the aldehyde functional group of PEG under such pegylation conditions. Thus, the present invention provides site-specific pegylation of the N-terminal cysteine residue. To prevent disulfide bridge formation during the pegylation, reducing agents such as tris(carboxyethyl)phosphine (TCEP) can be used and the reactions can be done under nitrogen and argon. 1-4 equivalents of PEG-aldehyde can be used. Reactions usually complete in 2 to 72 hours depending on the pH of the solution and the equivalents of PEG-aldehyde used. If the pegylation happens on unfolded proteins, the protein products can be refolded after pegylation. If the pegylation is done on correctly folded proteins, refolding step is omitted.

PEGs used in the present invention can have different molecular weights (e.g., 2-40 kDa), have linear, branched and multi-arm structures and contain one or more than one aldehyde functional group. When PEG containing two aldehyde functional groups is used, the final product will be protein or peptide dimer and the linker in between is the PEG. PEG with multiple aldehyde functional groups will generate multimer of pegylated proteins or peptides.

To control the pH of the reaction solution, buffered solution systems such as PBS can be used. The reaction solutions can also contain other agents such as EDTA to facilitate the reactions.

The final pegylated proteins and peptides can be purified by different purification methods such as reversed phase high performance liquid chromatography (RP-HPLC), size-exclusive chromatography, and ion-exchange chromatography, and characterized by MALDI-MS, chromatography methods, electrophoresis, amino acid analysis, and protein and peptide sequencing technologies.

In a first embodiment, the invention is directed to a method of chemically conjugating PEG containing a free aldehyde group to the unoxidized sulfhydryl side-chain and the free α-amino group of a cysteine residue of a molecule, said method comprising reacting the free aldehyde group of said PEG with the unoxidized sulfhydryl side-chain and the free α-amino group of said cysteine residue to generate a 1,3-thiazolidine group in a product, wherein said product has the structure of

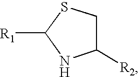

wherein R₁ is said PEG, and R₂ is said molecule.

In a second embodiment, the invention is directed to a method of chemically conjugating PEG containing a free aldehyde group to the unoxidized sulfhydryl side-chain and the free α-amino group of a cysteine residue of a molecule, said method comprising reacting the free aldehyde group of said PEG with the unoxidized sulfhydryl side-chain and the free α-amino group of said cysteine residue in a reaction solution to generate a 1,3-thiazolidine group in an intermediate, and adjusting the pH of the reaction solution to about 7, whereby said intermediate rearranges to form a final product, wherein said intermediate has the structure of

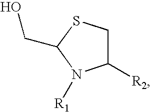

and said final product has the structure of

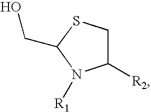

wherein R₁ is said PEG, and R₂ is said molecule. Here, the term “about” means±10%.

In a third embodiment, the invention is directed to a method of chemically conjugating PEG containing a free aldehyde group to the unoxidized sulfhydryl side-chain and the free α-amino group of a penicillamine residue of a molecule, said method comprising reacting the free aldehyde group of said PEG with the unoxidized sulfhydryl side-chain and the free α-amino group of said penicillamine residue to generate a 5,5-dimethyl-1,3-thiazolidine group in a product, wherein said product has the structure of

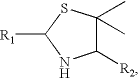

wherein R₁ is said PEG, and R₂ is said molecule.

In a fourth embodiment, the invention is directed to a method of chemically conjugating PEG containing a free aldehyde group to the unoxidized sulfhydryl side-chain and the free α-amino group of a penicillamine residue of a molecule, said method comprising reacting the free aldehyde group of said PEG with the unoxidized sulfhydryl side-chain and the free α-amino group of said penicillamine residue in a reaction solution to generate a 5,5-dimethyl-1,3-thiazolidine group in an intermediate, and adjusting the pH of the reaction solution to about 7, whereby said intermediate rearranges to form a final product, wherein said intermediate has the structure of

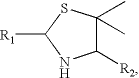

and said final product has the structure of

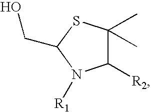

wherein R₁ is said PEG, and R₂ is said molecule. Here, the term “about” means±10%.

In a fifth embodiment, the invention is directed to a method of chemically conjugating PEG containing a free aldehyde group to the unoxidized sulfhydryl side-chain and the free α-amino group of a homocysteine residue of a molecule, said method comprising reacting the free aldehyde group of said PEG with the unoxidized sulfhydryl side-chain and the free α-amino group of said homocysteine residue to generate a six-membered ring system in a product, wherein said product has the structure of

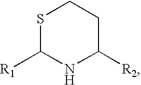

wherein R₁ is said PEG, and R₂ is said molecule.

In a sixth embodiment, the invention is directed to a method of chemically conjugating PEG containing a free aldehyde group to the unoxidized sulfhydryl side-chain and the free α-amino group of a homocysteine residue of a molecule, said method comprising reacting the free aldehyde group of said PEG with the unoxidized sulfhydryl side-chain and the free α-amino group of said homocysteine residue in a reaction solution to generate a six-membered ring system in an intermediate, and adjusting the pH of the reaction solution to about 7, whereby said intermediate rearranges to form a final product, wherein said intermediate has the structure of

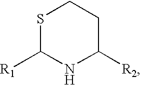

and said final product has the structure of

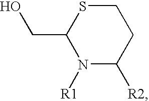

wherein R₁ is said PEG, and R₂ is said molecule. Here, the term “about” means±10%.

In a seventh embodiment, the invention is directed to a method of chemically conjugating PEG containing a free aldehyde group to the unoxidized free seleno group and the free α-amino group of a selenocysteine residue of a molecule, said method comprising reacting the free aldehyde group of said PEG with the unoxidized free seleno group and the free α-amino group of said selenocysteine residue to generate a five-membered ring system in a product, wherein said product has the structure of

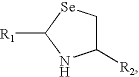

wherein R₁ is said PEG, and R₂ is said molecule.

In an eighth embodiment, the invention is directed to a method of chemically conjugating PEG containing a free aldehyde group to the unoxidized free seleno group and the free α-amino group of a selenocysteine residue of a molecule, said method comprising reacting the free aldehyde group of said PEG with the unoxidized free seleno group and the free α-amino group of said selenocysteine residue in a reaction solution to generate a five-membered ring system in an intermediate, and adjusting the pH of the reaction solution to about 7, whereby said intermediate rearranges to form a final product, wherein said intermediate has the structure of

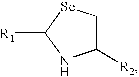

and said final product has the structure of

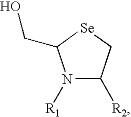

wherein R₁ is said PEG, and R₂ is said molecule. Here, the term “about” means±10%.

In a ninth embodiment, the invention is directed to a method of chemically conjugating PEG containing a free aldehyde group to the unoxidized sulfhydryl side-chain and the free α-methyl-amino group of an N-methyl-cysteine residue of a molecule, said method comprising reacting the free aldehyde group of said PEG with the unoxidized sulfhydryl side-chain and the free α-methyl-amino group of said N-methyl-cysteine residue to generate a 3-methyl-1,3-thiazolidine group in a product, wherein said product has the structure of

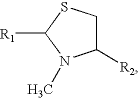

wherein R₁ is said PEG, and R₂ is said molecule.

In each of the foregoing embodiments of the invention—i.e., the first through ninth embodiments of the invention—the free aldehyde group is attached to said PEG through a linker that may contain amide, ester, sulfonamide, sulfonyl, thiol, oxy, alkyl, alkenyl, alkynyl, aryl, maleimide, or amine functional group, or any combination thereof.

In a tenth embodiment, the invention is directed to a method of chemically conjugating PEG containing a free maleimide group to the unoxidized sulfhydryl side-chain of an N-methyl-cysteine residue of a molecule, said method comprising reacting the free maleimide group of said PEG with the unoxidized sulfhydryl side-chain of said N-methyl-cysteine to generate a conjugate product, wherein said conjugate product has the structure of

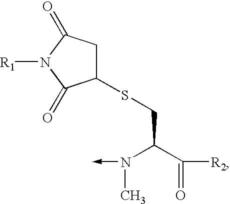

wherein R₁ is said PEG, and R₂ is said molecule.

In an eleventh embodiment, the invention is directed to a method of chemically conjugating PEG containing a free maleimide group to the unoxidized sulfhydryl side-chain of a penicillamine residue of a molecule, said method comprising reacting the free maleimide group of said PEG with the unoxidized sulfhydryl side-chain of said penicillamine residue to generate a conjugate product, wherein said conjugate product has the structure of

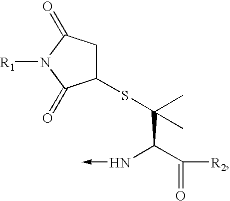

wherein R₁ is said PEG, and R₂ is said molecule.

In a twelfth embodiment, the invention is directed to a method of chemically conjugating PEG containing a free maleimide group to the unoxidized sulfhydryl side-chain of a homocysteine residue of a molecule, said method comprising reacting the free maleimide group of said PEG with the unoxidized sulfhydryl side-chain of said homocysteine residue to generate a conjugate product, wherein said conjugate product has the structure of

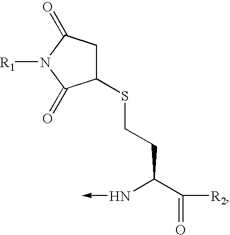

wherein R₁ is said PEG, and R₂ is said molecule.

In a thirteenth embodiment, the invention is directed to a method of chemically conjugating PEG containing a free maleimide group to the unoxidized seleno side-chain of a selenocysteine residue of a molecule, said method comprising reacting the free maleimide group of said PEG with the unoxidized seleno side-chain of said selenocysteine residue to generate a conjugate product, wherein said conjugate product has the structure of

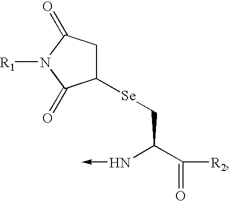

wherein R₁ is said PEG, and R₂ is said molecule.

In each of the foregoing embodiments of the invention—i.e., the tenth through thirteenth embodiments of the invention—the free maleimide group is attached to said PEG through a linker that may contain amide, ester, sulfonamide, sulfonyl, thiol, oxy, alkyl, alkenyl, alkynyl, aryl, maleimide, or amine functional group, or any combination thereof.

In all of the foregoing embodiments of the invention, the PEG may have a linear structure, a branched structure, or a multi-arm structure.

In all of the foregoing embodiments of the invention, the PEG has average molecular weight of about 100 Da to about 500,000 Da, and more preferably has average molecular weight of about 1,000 Da to about 50,000 Da.

### DETAILED DESCRIPTION OF THE INVENTION

It is believed that one skilled in the art can, based on the description herein, utilize the present invention to its fullest extent. The following specific embodiments are, therefore, to be construed as merely illustrative, and not limitative of the remainder of the disclosure in any way whatsoever.

Unless defined otherwise, all technical and scientific terms used herein have the same meaning as commonly understood by one of ordinary skill in the art to which this invention pertains. Also, all publications, patent applications, patents and other references mentioned herein are incorporated by reference, each in its entirety.

Nomenclature and Abbreviations

| Symbol | Meaning |
|---|---|
| Ala or A | alanine |
| Arg or R | arginine |
| Asn or N | asparagine |
| Asp or D | aspartic acid |
| Cys or C | cysteine |
| hCys | homocysteine |
| Gln or Q | glutamine |
| Glu or E | glutamic acid |
| Gly or G | glycine |
| His or H | histidine |
| Ile or I | isoleucine |
| Leu or L | leucine |
| Lys or K | lysine |
| Met or M | methionine |
| Nle | norleucine |
| N-Me-Cys or NMeCys | N-methyl-cysteine, which has the structure of |
| | 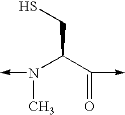 |
| PEG | polyethylene glycol |
| Pen | penicillamine |
| Phe or F | phenylalanine |
| Pro or P | proline |
| Ser or S | serine |
| selenoCys | selenocysteine |
| Thr or T | threonine |
| Trp or W | tryptophan |
| Tyr or Y | tyrosine |
| Val or V | valine |

### Certain Other Abbreviations Used Herein are Defined as Follows:

Boc tert-butyloxycarbonyl
Bzl benzyl
DCM dichloromethane
DIC N,N-diisopropylcarbodiimide
DIEA diisopropylethyl amine
Dmab 4-{N-(1-(4,4-dimethyl-2,6-dioxocyclohexylidene)-3-methylbutyl)-amino}benzyl
DMAP 4-(dimethylamino)pyridine
DMF dimethylformamide
DNP 2,4-dinitrophenyl
DTT dithiothreitol
EDTA ethylenediaminetetraacetic acid
Fmoc Fluorenylmethyloxycarbonyl
HBTU 2-(1H-benzotriazole-1-yl)-1,1,3,3-tetramethyluronium hexafluorophosphate
cHex cyclohexyl
HOAT O-(7-azabenzotriazol-1-yl)-1,1,3,3-tetramethyluronium hexafluorophosphate
HOBt 1-hydroxy-benzotriazole
Me methyl
Mmt 4-methoxytrityl
NMP N-methylpyrrolidone
Pbf 2,2,4,6,7-pentamethyldihydrobenzofuran-5-sulfonyl
tBu tert-butyl
TCEP tris(carboxyethyl)phosphine
TIS triisopropylsilane
TOS tosyl
trt trityl
TFA trifluoro acetic acid
TFFH tetramethylfluoroforamidinium hexafluorophosphate
Z benzyloxycarbonyl
Tha 1,3-thiazolidine-4-carboxylic acid, which has the structure of:

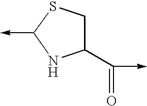

Tmc 1,3-thiazolidine-3-methyl-4-carboxylic acid, which has the structure of:

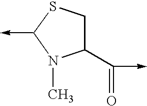

Dma 5,5-dimethyl-1,3-thiazolidine-4-carboxylic acid, which has the structure of:

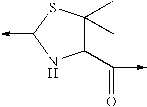

Thc 1,3-thiazinane-4-carboxylic acid, which has the structure of:

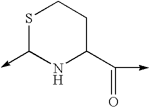

Sez 1,3-selenazolidine-4-carboxylic acid, which has the structure of:

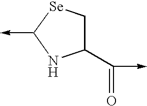

Hth 2-hydroxymethyl-1,3-thiazolidine-4-carboxylic acid, which has the structure of:

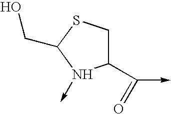

Hdm 2-hydroxymethyl-5,5-dimethyl-1,3-thiazolidine-4-carboxylic acid, which has the structure of:

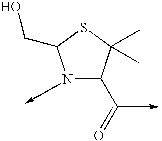

Haz 2-hydroxymethyl-1,3-thiazinane-4-carboxylic acid, which has the structure of:

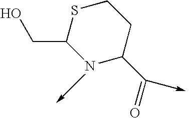

Hsz 2-hydroxymethyl-1,3-selenazolidine-4-carboxylic acid, which has the structure of:

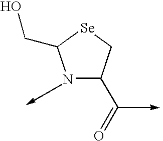

Maleimide has the structure of:

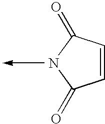

Prd pyrrolidine-2,5-dione, which has the structure of:

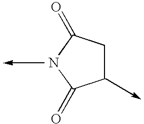

NMeCys(Prd-PEG) has the structure of:

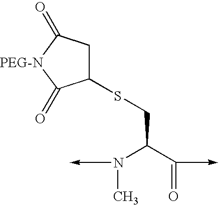

Pen(Prd-PEG) has the structure of:

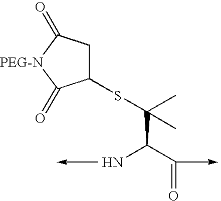

hCys(Prd-PEG) has the structure of:

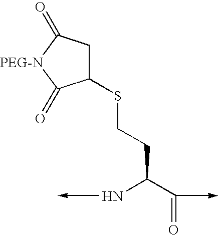

selenoCys(Prd-PEG) has the structure of:

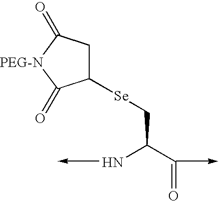

PEG is a well-known, water soluble polymer that is commercially available or can be prepared by ring-opening polymerization of ethylene glycol according to methods known in the art (Sandler and Karo, Polymer Synthesis, Academic Press, New York, VO13, pages 138-161). The term “PEG” is used broadly to encompass any polyethylene glycol molecule, without regard to size or to modification at end of the PEG. PEG may have linear, branched or multi-armed structure.

### EXAMPLES

### Example 1) Preparation of H-NMeCvs-Lvs-Phe-NH₂ (SEQ ID NO:1)

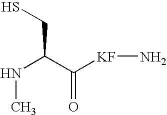

Rink amide MBHA resin (211 mg, 0.152 mmole) (Novabiochem, San Diego, Calif.) was swollen in dichloromethane (DCM) and washed with dimethylformamide (DMF). The resin was deblocked by treatment with a 25% piperidine/DMF (10 mL) solution for 2×10 min. The resin was washed with DMF (10 mL) three times. The first amino acid was coupled to the resin by treatment with a solution of Fmoc-Phe-OH (Novabiochem, San Diego, Calif.) (235 mg, 0.606 mmole), 1-hydroxybenzotriazole (HOBt) (92.3 mg, 0.606 mmole), and diisopropylcarbodiimide (DIC) (77 mg, 0.606 mmole) in N-methylpyrrolidone (NMP) (2 mL) for one hour. The resin was filtered and washed with DMF (10 mL) three times.

The Fmoc protecting group was removed by treatment with a 25% piperidine/DMF (10 mL) solution for 2×10 min and the resin was washed with DMF (1 mL) three times. Fmoc-Lys(Boc)-OH (Novabiochem, San Diego, Calif.) (285 mg 0.606 mmole) was coupled to the resulting free amine resin in the presence of HOBt (0.606 mmole) and DIC (0.606 mmole) in NMP (2 mL) for one hour.

The deblocking and washing procedures were repeated as above. Fmoc-N-Me-Cys(Trt)-OH (Timen Chemicals, Lodz, Poland.) (100 mg, 0.167 mmole) was coupled to the resulting peptide-resin by using HOBt (Slmg, 0.33 mmole) and DIC (83.8 mg, 0.66 mmole) in NMP (2 mL) for 12 hours. The coupling of Fmoc-N-Me-Cys(Trt)-OH (45 mg, 0.075 mmole) was repeated by using tetramethylfluoroformamidiniumpentafluorophosphate (TFFH) (20 mg, 0.075 mmole) and diisoproplyethylamine (DIEA) (19.4 mg, 0.150 mmole) in NMP (2 mL) for one hour. The deblocking and washing procedures were repeated as above. The resin was washed with DCM three times then with methanol three times. The resin was dried under vacuum.

The peptide was cleaved off from the resin by shaking the resin with 8% trispropylsilane/trifluoroacetic acid (TFA) (2 mL) for two hours. The resin was filtered and washed with DCM (2 mL). The filtrates were combined and concentrated to 1 mL. Diethyl ether (35 mL) was added to precipitate the peptide. The precipitated peptide was collected after centrifuging. The pellet was dissolved in water and acetonitrile and then was lyophilized.

The resulting crude product was purified on a reverse phase HPLC system (Luna 5 micron C8 (2) 10×20 mm column), eluted from 100% buffer A (0.1% TFA in water) and 0% buffer B (0.1% TFA in acetonitrile) to 80% buffer A and 20% buffer B over 30 minutes monitoring at 235 nm. After the lyophilization, 51.2 mg of the final product was obtained. An M+1 ion at 410.3 Da was detected by ESI mass spectroscopy, which is consistent with the calculated molecular weight of 409.6 Da.

### Example 2

### Preparation of mPEG-Tmc-Lvs-Phe-NH₂ (SEQ ID NO:2)

mPEG herein has the structure of CH₃—O—(CH₂CH₂O)ₙ—(CH₂)₂—, wherein n is a positive integer.

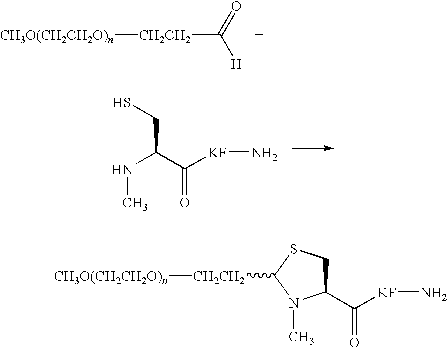

The peptide product of Example 1 (0.5 mg 1.22 micromole) was dissolved in 1.0 mL of a pH 4 buffer (20 mmolar NaOAc, 150 mmolar NaCl, and 1 mmolar EDTA). To the resulting solution was added mPEG-aldehyde (1.5 equivalents, the average molecular weight is 31378 Da, NOF Corp., Tokyo, Japan). The reaction was approximately 90% complete after 27 hours at room temperature based on the analysis done by using a reverse-phase analytical HPLC system (Vydac C₁₈ 5μ peptide/protein column, 4.6×250 mm). The reaction mixture was applied to a 5 mL Zeba™ desalt spin column (Pierce Biotechnology, Rockford, Ill.). A white foam was obtained after lyophilization (36.7 mg).

### Example 3

### Preparation of H-NMeCys(Prd-PEG)-Lys-Phe-NH₂ (SEQ ID NO:3)

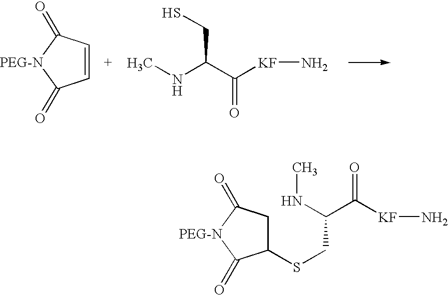

The peptide product of Example 1 (0.5 mg 1.22 micromole) was dissolved in 1.0 mL of a pH 7 buffer (20 mmolar NaOAc). To the resulting solution was added α-(3-(3-maleimido-1-oxopropyl)amino)propyl-o-methoxy-polyoxyethlene (1.5 equivalents, the average molecular weight is 11962 Da, NOF Corp., Tokyo, Japan) and 2 equivalents of Tris(2-carboxyethyl)phosphine hydrochloride (TCEP). The reaction was complete after one hour at room temperature based on the analysis done by using a reverse-phase analytical HPLC system (Vydac C₁₈ 5μ peptide/protein column, 4.6×250 mm). The reaction mixture was applied to a 5 mL Zeba™ desalt spin column (Pierce Biotechnology, Rockford, Ill.). A white foam was obtained after lyophilization (5.1 mg). The product was further purified on High Trap™ SPXL cation exchange column (GE Healthcare, Piscataway, N.J.). The molecular weight distribution of the purified product was determined by using MALDI-TOF mass spectroscopy. The obtained experimental result was consistent with the calculated molecular weight distribution.

### Example 4

### Preparation of H-Cys-Lys-Phe-NH₂ (SEQ ID NO:4)

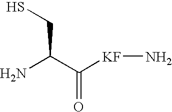

The title peptide was synthesized on a Liberty™ model microwave peptide synthesizer (CEM Corp., Matthews, N.C.) using Rink amide MBHA resin (347 mg 0.25 mmole) (Novabiochem, San Diego, Calif.). The amino acids Fmoc-Phe-OH, Fmoc Lys(Boc)-OH, and Fmoc-Cys(Trt)-OH (Novabiochem, San Diego, Calif.) were used in four fold excess using HBTU activation and each coupling was repeated.

The peptide was cleaved from the resin by shaking resin with 8% trispropylsilane/trifluoroacetic acid (TFA) with 1% dithiothreitol (10 mL) for three hours. The resin was filtered and washed with DCM (5 mL). The filtrates were combined and concentrated to 3 mL. Diethyl ether (35 mL) was added to precipitate the peptide. The precipitated peptide was collected after centrifuging. The pellet was dissolved in water and acetonitrile and then was lyophilized.

The resulting crude product was purified on a reverse phase HPLC system (Luna 5 micron C8 (2) 100×20 mm column), eluted from 100% buffer A (0.1% TFA in water) and 0% buffer B (0.1% TFA in acetonitrile) to 70% buffer A and 30% buffer B over 35 minutes monitoring at 235 nm. After the lyophilization, 89.1 mg of the final product was obtained. An M+1 ion at 396.5 Da was detected by ESI mass spectroscopy, which is consistent with the calculated molecular weight 395.5 Da.

### Example 5

### Preparation of mPEG-Tha-Lys-Phe-NH₂ (SEQ ID NO:5)

mPEG herein has the structure of CH₃—O—(CH₂CH₂O)ₙ—(CH₂)₂—, wherein n is a positive integer.

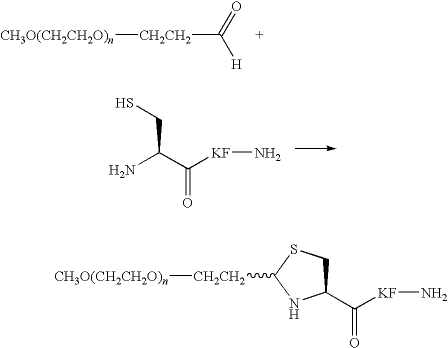

The peptide product of Example 4 (0.5 mg 1.26 micromole) was dissolved in 1.0 mL of a pH 4 buffer (20 mmolar NaOAc). To the resulting solution was added mPEG-aldehyde (1.5 equivalents, the average molecular weight is 20644 Da, NOF Corp., Tokyo, Japan) and TCEP (2.0 equivalents). The reaction was approximately 85% complete after three hours at room temperature based on the analysis done by using a reverse-phase analytical HPLC system (Vydac C₁₈ 5μ peptide/protein column, 4.6×250 mm). The reaction mixture was applied to a 10 mL Zeba™ desalt spin column (Pierce Biotechnology, Rockford, Ill.). A white foam was obtained after lyophilization.

### Example 6

### Preparation of H-hCys-Lys-Phe-NH₂ (SEQ ID NO:4)

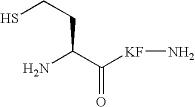

The title peptide was synthesized on a Liberty™ model microwave peptide synthesizer (CEM Corp., Matthews, N.C.) using Rink amide MBHA resin (347 mg 0.25 mmole) (Novabiochem, San Diego, Calif.). The amino acids Fmoc-Phe-OH, Fmoc Lys(Boc)-OH, and Fmoc-hCys(Trt)-OH (Novabiochem, San Diego, Calif.) were used in four fold excess using HBTU activation and each coupling was repeated.

The peptide was cleaved from the resin by shaking resin with 8% trispropylsilane/trifluoroacetic acid (TFA) with 1% dithiothreitol (10 mL) for three hours. The resin was filtered and washed with DCM (5 mL). The filtrates were combined and concentrated to 3 mL. Diethyl ether (35 mL) was added to precipitate the peptide. The precipitated peptide was collected after centrifuging. The pellet was dissolved in water and acetonitrile and then was lyophilized.

The resulting crude product was purified on a reverse phase HPLC system (Luna 5 micron C8 (2) 1 00×20 mm column), eluted from 100% buffer A (0.1% TFA in water) and 0% buffer B (0.1% TFA in acetonitrile) to 75% buffer A and 25% buffer B over 35 minutes monitoring at 235 nm. After the lyophilization, 85.7 mg of the final product was obtained. An M+1 ion at 410.5 Da was detected by ESI mass spectroscopy, which is consistent with the calculated molecular weight 409.6 Da.

### Example 7

### Preparation of H-Pen-Lys-Phe-NH₂ (SEQ ID NO:6)

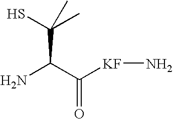

The title peptide was synthesized on a Liberty™ model microwave peptide synthesizer (CEM Corp., Matthews, N.C.) using Rink amide MBHA resin (347 mg 0.25 mmole) (Novabiochem, San Diego, Calif.). The amino acids Fmoc-Phe-OH, Fmoc Lys(Boc)-OH, and Fmoc-Pen(Trt)-OH (Novabiochem, San Diego, Calif.) were used in four fold excess using HBTU activation and each coupling was repeated.

The peptide was cleaved from the resin by shaking resin with 8% trispropylsilane/trifluoroacetic acid (TFA) with 1% dithiothreitol (10 mL) for three hours. The resin was filtered and washed with DCM (5 mL). The filtrates were combined and concentrated to 3 mL. Diethyl ether (35 mL) was added to precipitate the peptide. The precipitated peptide was collected after centrifuging. The pellet was dissolved in water and acetonitrile and then was lyophilized.

The resulting crude product was purified on a reverse phase HPLC system (Luna 5 micron C8 (2) 100×20 mm column), eluted from 100% buffer A (0.1% TFA in water) and 0% buffer B (0.1% TFA in acetonitrile) to 80% buffer A and 20% buffer B over 35 minutes monitoring at 235 nm. After the lyophilization, 83.9 mg of the final product was obtained. An M+1 ion at 424.5 Da was detected by ESI mass spectroscopy, which is consistent with the calculated molecular weight 423.6 Da.

### Example 8

### Preparation of mPEG-Dma-Lys-Phe-NH₂ (SEQ ID NO:7)

mPEG herein has the structure of CH₃—O—(CH₂CH₂O)ₙ—(CH₂)₂—, wherein n is a positive integer.

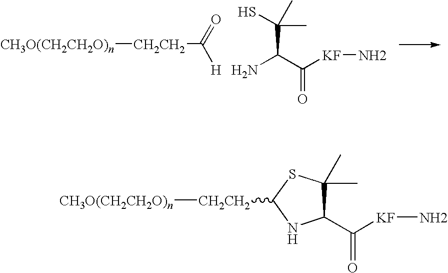

The peptide product of Example 7 (0.5 mg 1.18 micromole) was dissolved in 11.0 mL of a pH 4 buffer (20 mmolar NaOAc). To the resulting solution was added mPEG-aldehyde (1.5 equivalents, the average molecular weight is 20644 Da, NOF Corp., Tokyo, Japan) and TCEP (2.0 equivalents). The reaction was approximately 80% complete after three hours at room temperature based on the analysis done by using a reverse-phase analytical HPLC system (Vydac C₁₈ 5μ peptide/protein column, 4.6×250 mm). The reaction mixture was applied to a 10 mL Zeba™ desalt spin column (Pierce Biotechnology, Rockford, Ill.). A white foam was obtained after lyophilization.

### Example 9

### Preparation of mPEG-Thc-Lys-Phe-NH₂ (SEQ ID NO:8)

mPEG herein has the structure of CH₃—O—(CH₂CH₂O)ₙ—(CH₂)₂—, wherein n is a positive integer.

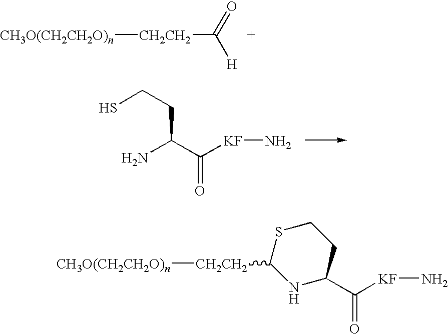

The peptide product of Example 6 (0.5 mg 1.22 micromole) was dissolved in 11.0 mL of a pH 4 buffer (20 mmolar NaOAc). To the resulting solution was added mPEG-aldehyde (1.5 equivalents, the average molecular weight is 20644 Da, NOF Corp., Tokyo, Japan) and TCEP (2.0 equivalents). The reaction was approximately 90% complete after three hours at room temperature based on the analysis done by using a reverse-phase analytical HPLC system (Vydac C₁₋₈ 5μ peptide/protein column, 4.6×250 mm). The reaction mixture was applied to a 10 mL Zeba™ desalt spin column (Pierce Biotechnology, Rockford, Ill.). A white foam was obtained after lyophilization

### Example 10

### Preparation of selenoCys-Lys-Phe-NH₂ (SEQ ID NO:9)

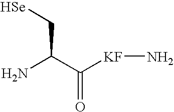

The title peptide is synthesized substantially according to the procedure described in Example 1. Fmoc-selenoCys(4-MeOBzl)-OH (Novabiochem, San Diego, Calif.) is used for the incorporation of selenocysteine residue at the N-terminus.

### Example 11

### Preparation of mPEG-Sez-Lys-Phe-NH₂ (SEQ ID NO:10)

mPEG herein has the structure of CH₃—O—(CH₂CH₂O)ₙ—(CH₂)₂—, wherein n is a positive integer.

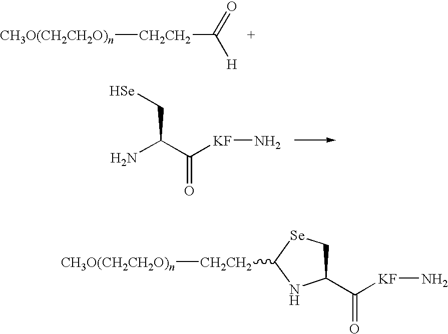

The title peptide is synthesized substantially according to the procedure described in Example 2. The product obtained from Example 10 is the peptide starting material.

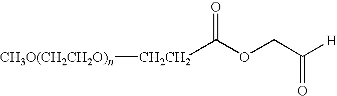

### Example 12

### Preparation of

mPEG herein has the structure of CH₃—O—(CH₂CH₂O)ₙ—(CH₂)₂—, wherein n is a positive integer.

mPEG-C(O)OH cesium salt reacts with bromoacetaldehyde dimethyl acetal in DMF at 60° C. for 2 days. After removing the solvent, the product is treated with 40% TFA in DCM with small amount of water at 0° C. for about 30 min.

### Example 13

### Preparation of mPEG-Hth-Lys-Phe-NH₂ (SEQ ID NO:11)

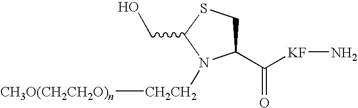

The mPEG herein has the structure of CH₃—O—(CH₂CH₂O)ₙ—(CH₂)₂—, wherein n is a positive integer.

The title peptide is synthesized substantially according to the procedure described in Example 2. The peptide starting material is the product obtained from Example 4. The PEG-aldehyde starting material is the product obtained in Example 12. There is an additional step of adjusting pH of the buffer solution: after standing at room temperature for 2 hours at pH4, the pH of the reaction solution is adjusted to 7 and stands at room temperature for 3 days before purification.

### Example 14

### Preparation of mPEG-Hdm-Lys-Phe-NH₂ (SEQ ID NO:12)

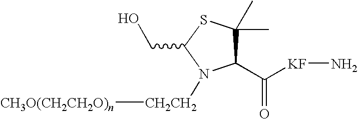

The mPEG herein has the structure of CH₃—O—(CH₂CH₂O)ₙ—(CH₂)₂—, wherein n is a positive integer.

The title peptide is synthesized substantially according to the procedure described for Example 8. The peptide starting material is the product obtained from Example 7. The PEG-aldehyde starting material is the product obtained in Example 12. There is an additional step of adjusting pH of the buffer solution: after standing at room temperature overnight, the pH of the reaction solution is adjusted to 7 and the solution stands at room temperature for 3 days before purification.

### Example 15

### Preparation of mPEG-Haz-Lys-Phe-NH₂ (SEQ ID NO:13)

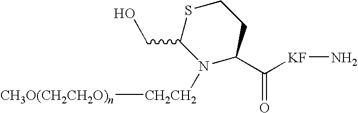

The mPEG herein has the structure of CH₃—O—(CH₂CH₂O)ₙ—(CH₂)₂—, wherein n is a positive integer.

The title peptide is synthesized substantially according to the procedure described for Example 9. The peptide starting material is the product obtained from Example 6. The PEG-aldehyde starting material is the product obtained in Example 12. There is an additional step of adjusting pH of the buffer solution: after standing at room temperature overnight at pH4, the pH of the reaction solution is adjusted to 7 and the solution stands at room temperature for 3 days before purification.

### Example 16

### Preparation of mPEG-Hsz-Lys-Phe-NH₂ (SEQ ID NO:14)

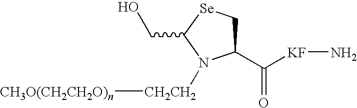

The mPEG herein has the structure of CH₃—O—(CH₂CH₂O)ₙ—(CH₂)₂—, wherein n is a positive integer.

The title peptide is synthesized substantially according to the procedure described for Example 11. The peptide starting material is the product obtained from Example 10. The PEG-aldehyde starting material is the product obtained in Example 12. There is an additional step of adjusting pH of the buffer solution: after standing at room temperature for 2 hours at pH4, the pH of the reaction solution is adjusted to 7 and the solution stands at room temperature for 3 days before purification.

### Example 17

### Preparation of H-Pen(Prd-PEG)-Lys-Phe-NH₂ (SEQ ID NO:15)

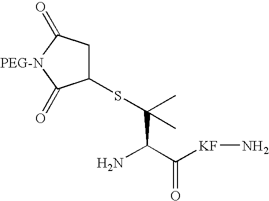

The title peptide is synthesized substantially according to the procedure described in Example 3. The peptide starting material is the product obtained from Example 7.

### Example 18

### Preparation of H-hCys(Prd-PEG)-Lys-Phe-NH₂ (SEQ ID NO:16)

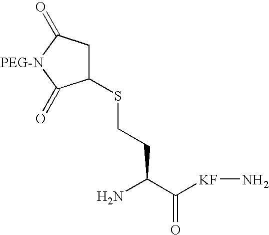

The peptide product of Example 6 (11.0 mg 2.44 micromole) was dissolved in 11.0 mL of a pH 7 buffer (20 mmolar NaOAc). To the resulting solution was added α-(3-(3-maleimido-1-oxopropyl)amino)propyl-ω-methoxy-polyoxyethlene (1.5 equivalents, the average molecular weight is 11962 Da, NOF Corp., Tokyo, Japan) and 2 equivalents of Tris(2-carboxyethyl)phosphine hydrochloride (TCEP). The reaction was complete after one hour at room temperature based on the analysis done by using a reverse-phase analytical HPLC system (Vydac C₁₈ 5μ peptide/protein column, 4.6×250 mm). The reaction mixture was applied to a 10 mL Zeba™ desalt spin column (Pierce Biotechnology, Rockford, Ill.). A white foam was obtained after lyophilization.

### Example 19

### Preparation of H-selenoCys(Prd-PEG)-Lys-Phe-NH₂ (SEQ ID NO:17)

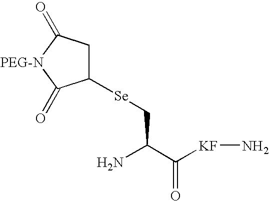

The title peptide is synthesized substantially according to the procedure described in Example 3. The peptide starting material is the product obtained from Example 10.

1. A method of chemically conjugating PEG containing one or more a free aldehyde groups to the unoxidized sulfhydryl side-chain and the free α-amino group of a D- or L-cysteine residue of a molecule, said method comprising reacting the free aldehyde group of said PEG with the unoxidized sulfhydryl side-chain and the free α-amino group of said cysteine residue to generate a 1,3-thiazolidine group in a product, wherein said product has the structure of 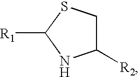
wherein R₁ is said PEG, and R₂ is said molecule.

2. A method of chemically conjugating PEG containing one or more a free aldehyde groups group to the unoxidized sulfhydryl side-chain and the free α-amino group of a D- or L-cysteine cysteine residue of a molecule according to claim 1, wherein said method further comprises
adjusting the pH of the reaction solution to about 7, whereby said intermediate rearranges to form a final product, wherein said intermediate has the structure of 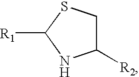
and said final product has the structure of 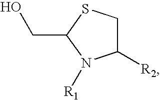
wherein R₁ is said PEG, and R₂ is said molecule.

3-18. (canceled)

19. A method of chemically conjugating PEG containing one or more a free aldehyde to the unoxidized sulfhydryl side-chain and the free α-amino group of a D- or L-penicillamine residue of a molecule, said method comprising reacting the free aldehyde group of said PEG with the unoxidized sulfhydryl side-chain and the free α-amino group of said penicillamine residue to generate a 5,5-dimethyl-1,3-thiazolidine group in a product, wherein said product has the structure of 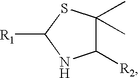
wherein R₁ is said PEG, and R₂ is said molecule.

20. A method of chemically conjugating PEG containing one or more a free aldehyde groups to the unoxidized sulfhydryl side-chain and the free α-amino group of a D- or L-penicillamine residue of a molecule according to claim 19, wherein said method further comprises
adjusting the pH of the reaction solution to about 7, whereby said intermediate rearranges to form a final product, wherein said intermediate has the structure of 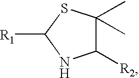
and said final product has the structure of 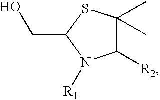
wherein R₁ is said PEG, and R₂ is said molecule.

21-36. (canceled)

37. A method of chemically conjugating PEG containing one or more a free aldehyde groups to the unoxidized sulfhydryl side-chain and the free α-amino group of a D- or L-homocysteine residue of a molecule, said method comprising reacting the free aldehyde group of said PEG with the unoxidized sulfhydryl side-chain and the free α-amino group of said homocysteine residue to generate a six-membered ring system in a product, wherein said product has the structure of 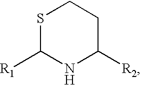
wherein R₁ is said PEG, and R₂ is said molecule.

38. A method of chemically conjugating PEG containing one or more a free aldehyde groups to the unoxidized sulfhydryl side-chain and the free α-amino group of a D- or L-homocysteine residue of a molecule according to claim 37, wherein said method further comprises,
adjusting the pH of the reaction solution to about 7, whereby said intermediate rearranges to form a final product, wherein said intermediate has the structure of 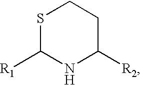
and said final product has the structure of 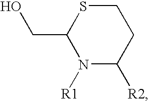
wherein R₁ is said PEG, and R₂ is said molecule.

39-54. (canceled)

55. A method of chemically conjugating PEG containing one or more a free aldehyde groups to the unoxidized free seleno group and the free α-amino group of a D- or L-selenocysteine residue of a molecule, said method comprising reacting the free aldehyde group of said PEG with the unoxidized free seleno group and the free α-amino group of said selenocysteine residue to generate a five-membered ring system in a product, wherein said product has the structure of 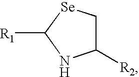
wherein R₁ is said PEG, and R₂ is said molecule.

56. A method of chemically conjugating PEG one or more a free aldehyde groups to the unoxidized free seleno group and the free α-amino group of a D- or L-selenocysteine residue of a molecule according to claim 55, wherein said method further comprises,
adjusting the pH of the reaction solution to about 7, whereby said intermediate rearranges to form a final product, wherein said intermediate has the structure of 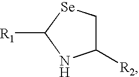
and said final product has the structure of 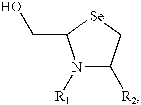
wherein R₁ is said PEG, and R₂ is said molecule.

57-72. (canceled)

73. A method of chemically conjugating PEG containing one or more a free aldehyde groups the unoxidized sulfhydryl side-chain and the free α-methyl-amino group of a D- or L-N-methyl-cysteine residue of a molecule, said method comprising reacting the free aldehyde group of said PEG with the unoxidized sulfhydryl side-chain and the free α-methyl-amino group of said N-methyl-cysteine residue to generate a 3-methyl-1,3-thiazolidine group in a product, wherein said product has the structure of 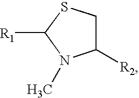
wherein R₁ is said PEG, and R₂ is said molecule.

74-88. (canceled)

89. A method of chemically conjugating PEG containing one or more a free maleimide groups to the unoxidized sulfhydryl side-chain of a D- or L-N-methyl-cysteine residue of a molecule, said method comprising reacting the free maleimide group of said PEG with the unoxidized sulfhydryl side-chain of said N-methyl-cysteine to generate a conjugate product, wherein said conjugate product has the structure of 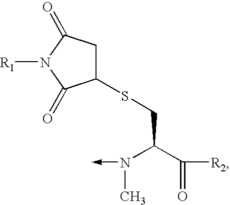
wherein R₁ is said PEG, and R₂ is said molecule.

90-104. (canceled)

105. A method of chemically conjugating PEG containing one or more a free maleimide groups group to the unoxidized sulfhydryl side-chain of a D- or L-penicillamine residue of a molecule, said method comprising reacting the free maleimide group of said PEG with the unoxidized sulfhydryl side-chain of said penicillamine residue to generate a conjugate product, wherein said conjugate product has the structure of 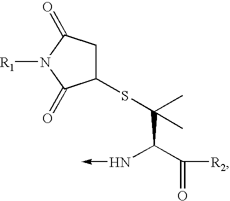
wherein R₁ is said PEG, and R₂ is said molecule.

106-120. (canceled)

121. A method of chemically conjugating PEG containing one or more a free maleimide groups to the unoxidized sulfhydryl side-chain of a D- or L-homocysteine residue of a molecule, said method comprising reacting the free maleimide group of said PEG with the unoxidized sulfhydryl side-chain of said homocysteine residue to generate a conjugate product, wherein said conjugate product has the structure of 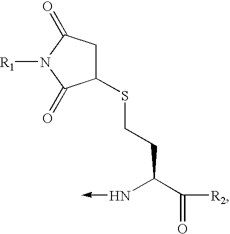
wherein R₁ is said PEG, and R₂ is said molecule.

122-136. (canceled)

137. A method of chemically conjugating PEG containing one or more a free maleimide groups to the unoxidized seleno side-chain of a D- or L-selenocysteine residue of a molecule, said method comprising reacting the free maleimide group of said PEG with the unoxidized seleno side-chain of said selenocysteine residue to generate a conjugate product, wherein said conjugate product has the structure of 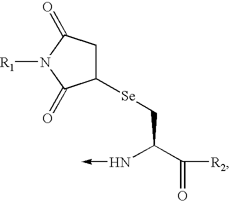
wherein R₁ is said PEG, and R₂ is said molecule.

138. The method according to any one of claims 1, 2, 19, 20, 37, 38, 55, 56, 73, 89, 105, 121, and 137, wherein the free aldehyde or maleimide group is attached to said PEG through a linker that may contain amide, ester, sulfonamide, sulfonyl, thiol, oxy, alkyl, alkenyl, alkynyl, aryl, maleimide, or amine functional group, or any combination thereof.

139. The method according to claim 137, wherein said PEG has a linear, branched or multi-arm structure.

140-142. (canceled)

143. The method according to claim 139, wherein said PEG has average molecular weight of about 100 Da to about 500,000 Da, preferably about 1,000 Da to about 50,000 Da.

144-146. (canceled)

147. The method according to any one of claims 1, 2, 19, 20, 37, 38, 55, 56, 73, 89, 105, 121, and 137 claim 137, wherein said cysteine, penicillamine, homocystene, selenocysteine, N-methyl-cysteine or maleimide residue is in an organic molecule, preferably a protein or a peptide.

148-149. (canceled)

150. The method according to any one of claims 1, 2, 19, 20, 37, 38, 55, 56, 73, 89, 105, 121, and 137 claim 137, wherein said conjugate product contains one or more protein, peptide, or organic molecule moieties, or any combination thereof.

151. The method according to any one of claims 1, 2, 19, 20, 37, 38, 55, 56, 73, 89, 105, 121, and 137 claim 137, wherein a reducing agent is used in the reaction.

152. The method according to claim 151, wherein said reducing agent is selected from the group consisting of TCEP, compounds containing an unoxidized sulfhydryl and compounds containing an unoxidized free seleno group.


SEQUENCE LISTING (17 sequences)

SEQ ID NO: 1 - protein, 3 aa, Artificial Sequence
  Peptide for site-specific pegylation
XKF

SEQ ID NO: 2 - protein, 2 aa, Artificial Sequence
  Pegylated peptide
XF

SEQ ID NO: 3 - protein, 3 aa, Artificial Sequence
  Pegylated peptide
XKF

SEQ ID NO: 4 - protein, 3 aa, Artificial Sequence
  Peptide for site-specific pegylation
XKF

SEQ ID NO: 5 - protein, 2 aa, Artificial Sequence
  Pegylated peptide
XF

SEQ ID NO: 6 - protein, 2 aa, Artificial Sequence
  Peptide for site-specific pegylation
XF

SEQ ID NO: 7 - protein, 2 aa, Artificial Sequence
  Pegylated peptide
XF

SEQ ID NO: 8 - protein, 2 aa, Artificial Sequence
  Pegylated peptide
XF

SEQ ID NO: 9 - protein, 3 aa, Artificial Sequence
  Peptide for site-specific pegylation
XKF

SEQ ID NO: 10 - protein, 2 aa, Artificial Sequence
  Pegylated peptide
XF

SEQ ID NO: 11 - protein, 2 aa, Artificial Sequence
  Pegylated peptide
XF

SEQ ID NO: 12 - protein, 2 aa, Artificial Sequence
  Pegylated peptide
XF

SEQ ID NO: 13 - protein, 2 aa, Artificial Sequence
  Pegylated peptide
XF

SEQ ID NO: 14 - protein, 2 aa, Artificial Sequence
  Pegylated peptide
XF

SEQ ID NO: 15 - protein, 2 aa, Artificial Sequence
  Pegylated peptide
XF

SEQ ID NO: 16 - protein, 3 aa, Artificial Sequence
  Pegylated peptide
XKF

SEQ ID NO: 17 - protein, 3 aa, Artificial Sequence
  Pegylated peptide
XKF